(19) United States
(12) Patent Application Publication
(10) Pub. No.: US 2026/0007340 A1
(43) Pub. Date: Jan. 8, 2026

(54) DEVICES AND METHODS FOR GAS ANALYSIS OF BODILY FLUIDS OR TISSUES

(71) Applicant: TheraNova, LLC, San Francisco, CA (US)

(72) Inventor: Daniel R. BURNETT, San Francisco, CA (US)

(73) Assignee: TheraNova, LLC, San Francisco, CA (US)

(21) Appl. No.: 19/323,266

(22) Filed: Sep. 9, 2025

Related U.S. Application Data
(63) Continuation of application No. PCT/US2024/019376, filed on Mar. 11, 2024.
(60) Provisional application No. 63/451,567, filed on Mar. 11, 2023.

(51) Int. Cl. A61B 5/1473; A61B 5/00; A61B 5/145

(52) U.S. Cl. CPC A61B 5/1473; A61B 5/14542; A61B 5/6852

(57) ABSTRACT

Catheter based devices and methods for gas analysis of bodily fluids or tissues are described. The system generally comprises a catheter having a length, one or more membranes positioned along the length, wherein the one or more membranes are configured to diffuse one or more compounds from a fluid, bodily gas, or tissue of interest through the one or more membranes and into a membrane space defined by the at least one membrane. A gas introduction lumen may be in fluid communication with the membrane space for introducing a gas. A gas extraction lumen may be in fluid communication with the membrane space for extracting the gas and the one or more compounds dispersed within the gas from the membrane space. An analyzer may be in communication with the membrane space, wherein the analyzer is configured to determine a parameter of the one or more compounds.

### CROSS-REFERENCE TO RELATED APPLICATIONS

This application is a continuation of International App. PCT/US2024/019376 filed Mar. 11, 2024, which claims the benefits of priority to U.S. Prov. 63/451,567 filed Mar. 11, 2023, which is incorporated herein by reference in its entirety.

### BACKGROUND OF THE INVENTION

The present invention relates to the field of analysis of gases or other components in bodily fluid or tissues using a modified catheter.

Catheters, such as PICC catheters, central catheters, Foley catheters, IV catheters, dialysis catheters, or other catheters may be placed in the body, such as in the vasculature, the bladder, the brain, the stomach, the peritoneal cavity or anywhere in the body. It would be desirable to use these catheters to analyze the bodily tissues, gas or fluids surrounding them while maintaining the functionality of the catheter.

### SUMMARY OF THE INVENTION

Disclosed herein are embodiments of analysis catheters systems and methods which include a catheter with a gas permeable membrane. The catheter system may be used to collect and analyze components in a body, including components in blood, gas and/or tissue. Embodiments of the catheter include a gas permeable section, such as ePTFE, or other suitable material. The membrane may or may not be impermeable to liquids. The membrane may be permeable to some gasses and not others or specific liquids or solid particles.

For example, the membrane may be permeable to O₂ (Oxygen) and/or CO₂ (Carbon Dioxide) and or N₂ (Nitrogen) and impermeable to blood and/or other liquids. The membrane may be specific to any component, including any gas or any other component. A neutral gas, such as air, may be introduced into the catheter through an entry lumen. The neutral gas may be introduced into a space within the membrane area. The membrane space may comprise an annular space between the membrane and the outer surface of the catheter shaft. The membrane allows the component to be analyzed, for example CO₂ or O₂, to cross the membrane and equilibrate in concentration with the gas in the space within the membrane area. The gas may then be extracted via an extraction lumen in the catheter. The extracted gas may be analyzed for the component of interest. The amount, or concentration, of component analyzed is indicative of the amount, or concentration, of the component in the body in the immediate area surrounding the membrane area of the catheter.

For example, the neutral gas may be air, the component of interest may be CO₂, and the catheter may be placed within a blood vessel. The CO₂ in the blood will cross the membrane of the catheter and the concentration of CO₂ within the space of the membrane area will equilibrate with the concentration of CO₂ in the blood surrounding the membrane area of the catheter. The gas may be extracted and analyzed for CO₂ concentration. The concentration of the CO₂ in the extracted gas is indicative of the CO₂ concentration of the blood. In order to speed the gas transfer, the initial gas that is pumped into the catheter may be different than the gas measured. For example in one embodiment, such gas can be an inert gas such as Nitrogen. The gas pumped can gradually be replaced with the gas measured once equilibrium is met. The replacement can be performed by gradually changing the concentration of the components. For example, air will be adjusted to reach the level of oxygen in the arteries which is 75 to 100 millimeters of mercury (mm Hg).

The system may include a controller. The controller may control a pump to pump the neutral gas into the membrane space. The same pump, or a different pump may be used to extract the gas containing the component. Alternatively, the extracted gas may exit naturally based on the positive pressure created by the neutral gas or negative pressure created by the suction of the measured gas. The advantage of such system will be that there no risk of introducing gas that will escape the catheter into the body. Further to that, a check valve also referred to as a backflow prevention valve or a one way valve may be installed at the tip of the catheter to prevent liquids inserting the membrane cavity when such positive pressure is applied, or preventing gas from entering the body when positive pressure is applied thus improving the safety and performance of the lumens. Alternatively, the pump may alternate between sending liquids through the catheter and extracting the gas form the exit lumen. In a different embodiment, the pump may temporally alternate between introducing gas and extracting gas to and from the catheter.

The neutral gas may be introduced and extracted periodically or continually. In some embodiments, the introduced gas may not be neutral, and may contain elements that may interact with the measured component or other components.

In another embodiment, the membrane will be impermeable to allow certain liquids to pass through. This process is also referred to as micro dialysis. In such system the inserted liquid may have specific components that may chemically interact with the permeated components to create a different component that may be analyzed. In the case of a semi-permeable membrane that allows liquids to pass through, the mechanism can be activated via hydro pressure, chemical osmosis or electrical gradient.

In yet another embodiment the catheter may also include a probe to measure the pH, a probe to measure the core temperature, and the internal blood pressure. The probe may have multiple membranes. The additional membrane can act as an ion transfer to measure salts (such as sodium or potassium).

The membranes may be designed in a concentric manner wherein the outer membrane may be semipermeable and will allow liquids to pass through. In such case, the Ph can be measured on the liquids. The second membrane may be permeable only to selected gases.

In yet another embodiment of the catheter based system for analyzing one or more components in a body, the system may generally comprise a catheter having a length, one or more membranes positioned along the length of the catheter, wherein the one or more membranes are configured to diffuse one or more compounds from a fluid, bodily gas, or tissue of interest through the one or more membranes and into a membrane space defined by the at least one membrane, a gas introduction lumen in fluid communication with the membrane space for introducing a gas through the length of the catheter and into the membrane space, a gas extraction lumen in fluid communication with the membrane space for extracting the gas and the one or more compounds dispersed within the gas from the membrane space, and an analyzer in communication with the membrane space, wherein the analyzer is configured to determine a parameter of the one or more compounds.

In another aspect of the catheter based system, the system may further comprise a pump in communication with the gas introduction lumen.

In another aspect of the catheter based system, the one or more membranes may be configured for contacting blood.

In another aspect of the catheter based system, the one or more membranes may be configured to be selectively permeable to one or more types of compounds.

In another aspect of the catheter based system, the one or more types of compounds comprise oxygen, carbon dioxide, or nitrogen.

In another aspect of the catheter based system, the analyzer may be configured to determine a concentration of the one or more compounds.

In another aspect of the catheter based system, the analyzer may be configured to determine information relating to arterial blood gases.

In another aspect of the catheter based system, the system may further comprise at least one additional membrane which defines the membrane space.

In another aspect of the catheter based system, the membrane space defines an annular space within or around the catheter.

In another aspect of the catheter based system, the annular space may be defined through the length of the catheter from the membrane space and to the analyzer.

In another aspect of the catheter based system, the pump may be configured to form an adjustable negative pressure within the membrane space.

In another aspect of the catheter based system, the system may further comprise a one-way valve near or at a tip of the catheter, wherein the valve is configured to prevent gas from entering the body.

In another aspect of the catheter based system, the pump may be configured to pump the gas through the length of the catheter and into the membrane space.

In another aspect of the catheter based system, the gas may comprise an inert gas.

In another aspect of the catheter based system, the pump may be configured to pump air with adjustable oxygen and nitrogen concentration.

In another aspect of the catheter based system, the one or more membranes may comprise a directional membrane configured to allow for unidirectional movement of the one or more compounds through the one or more membranes and into the membrane space.

In another aspect of the catheter based system, the directional membrane may be configured to allow for bidirectional movement of the one or more compounds.

In another aspect of the catheter based system, the system may further comprise one or more sensors positioned along the catheter and configured to measure pH or core temperature.

In another aspect of the catheter based system, any of the individual features above may be combined in any number of combinations and such combinations are intended to be fully within the scope of this disclosure.

In yet another embodiment of the method of analyzing one or more components in a body, the method may generally comprise positioning a catheter into contact with a fluid or tissue of interest within the body, wherein the catheter comprises one or more membranes which defines a membrane space within, introducing a gas through a length of the catheter and into the membrane space such that one or more compounds which have diffused through the one or more membranes from the fluid or tissue of interest and into the membrane space are dispersed within the gas, extracting the gas having the one or more compounds from the membrane space and into an analyzer in fluid communication with the membrane space, and determining a parameter of the one or more compounds via the analyzer.

In another aspect of the method of analyzing one or more components in the body, the method may comprise positioning the one or more membranes into contact with blood.

In another aspect of the method of analyzing one or more components in the body, the one or more membranes may be configured to be selectively permeable to one or more types of compounds.

In another aspect of the method of analyzing one or more components in the body, the one or more types of compounds comprise oxygen, carbon dioxide, or nitrogen.

In another aspect of the method of analyzing one or more components in the body, determining the parameter may comprise determining a concentration of the one or more compounds.

In another aspect of the method of analyzing one or more components in the body, determining the parameter may comprise determining information relating to arterial blood gases. This may include additional sensors that contact the body fluids to measure the pH. Furthermore, additional one or more sensors may be positioned to measure the blood pressure where systolic, diastolic or both.

In another aspect of the method of analyzing one or more components in the body, the catheter may further comprise at least one additional membrane which defines the membrane space.

In another aspect of the method of analyzing one or more components in the body, the membrane space may define an annular space within or around the catheter.

In another aspect of the method of analyzing one or more components in the body, the annular space may be defined through a length of the catheter from the membrane space and to the analyzer.

In another aspect of the method of analyzing one or more components in the body, adjusting the concentration of the gas until the one or more compounds are in equilibrium through the one or more membranes.

In another aspect of the method of analyzing one or more components in the body, extracting the gas having the one or more compounds may comprise withdrawing the gas via an adjustable negative pressure formed within the membrane space to prevent gas entering the body through said catheter.

In another aspect of the method of analyzing one or more components in the body, introducing the gas through the length of the catheter may comprise pumping the gas into the membrane space.

In another aspect of the method of analyzing one or more components in the body, pumping the gas into the membrane space may comprise pumping air in adjustable concentration of oxygen, carbon dioxide, and nitrogen into the membrane space.

In another aspect of the method of analyzing one or more components in the body, the one or more membranes may comprise a directional membrane configured to allow for unidirectional movement of the one or more compounds through the one or more membranes and into the membrane space.

In another aspect of the method of analyzing one or more components in the body, the directional membrane may be configured to allow for bidirectional movement of the one or more compounds.

In another aspect of the method of analyzing one or more components in the body, the method may further comprise sensing a core temperature via one or more sensors positioned along the catheter.

In another aspect of the method of analyzing one or more components in the body, the method may further comprise sensing a pH via one or more sensors positioned along the catheter.

In another aspect of the method of analyzing one or more components in the body, the method may further comprise sensing systolic and diastolic blood pressure via one or more sensors positioned along the catheter.

In another aspect of the method of analyzing one or more components in the body, a pump may temporally alternate between introducing a gas and extracting a gas.

In another aspect of the method of analyzing one or more components in the body, the gas may contain one or more elements that interact with the measured component or other components.

In another aspect of the method, any of the individual features above may be combined in any number of combinations and in any order and such combinations are intended to be fully within the scope of this disclosure.

### BRIEF DESCRIPTION OF THE DRAWINGS

FIG. 1 shows an embodiment of the analyzing catheter.

FIGS. 2A-2E show cross sectional views of the analyzing catheter at the indicated locations in FIG. 1.

FIG. 3 shows another embodiment of the analyzing catheter.

FIGS. 4A-4D show cross sectional views of the analyzing catheter at the

indicated locations in FIG. 3.

FIG. 5 shows the analyzing catheter system.

FIG. 6 shows an example of data processing system.

### DETAILED DESCRIPTION OF THE INVENTION

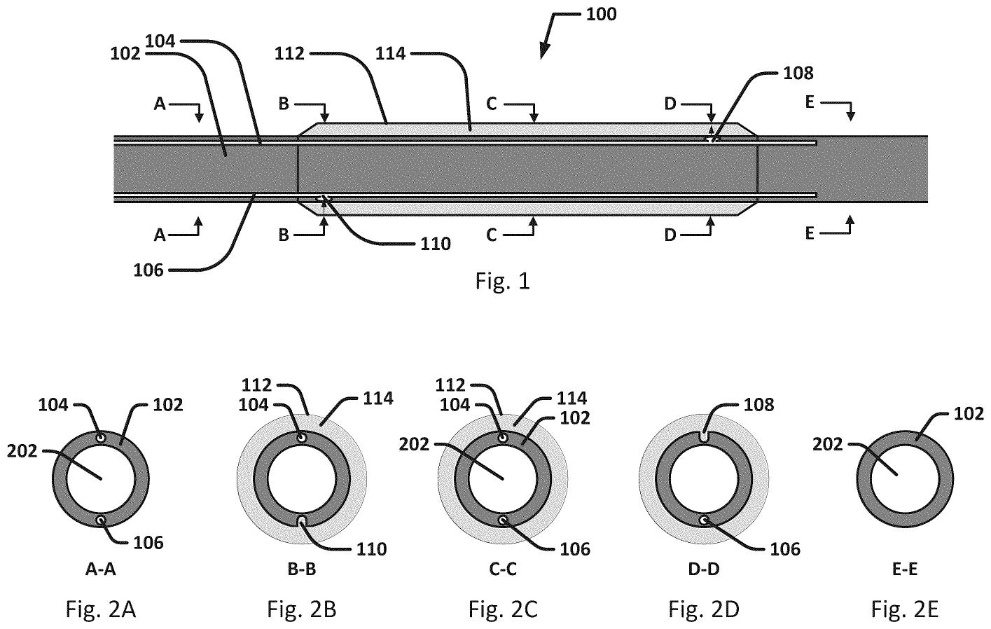

FIG. 1 shows an embodiment of the analyzing catheter. Analyzing catheter 100 includes catheter shaft 102, gas entry lumen 104, gas exit lumen 106, gas entry opening 108, gas exit opening 110, membrane 112 and membrane space 114. Gas, such as a neutral gas, or air, is intruded into membrane space 114 via entry lumen 104 and entry opening 108. The gas within membrane space 114 is exposed to fluid or tissue in the area of the membrane (e.g. blood, in a blood vessel). Compounds which are small enough to cross the membrane cross from the blood or fluid or tissue into annular membrane space 114. The concentration of the compound will equilibrate across the membrane. The gas containing the compound is extracted via gas exit opening 110 and gas exit lumen 106 and analyzed to determine the concentration of the compound in the gas. From the information the controller may determine the concentration of the compound in the bodily fluid and/or tissue, such as the blood. The concentration of the compound may be the same in the extracted gas and the blood, or it may be different in a predictable way so that the controller can determine the concentration of the compound in the blood.

The membrane 112 may comprise of multiple layers one allowing liquids to pass through and the other allowing only gas to pass through. The area between the two membranes can be used to position a Ph probe into the liquid. The time reaching equilibrium pay also be used to calculate the various concentrations of the measured compounds. In a different embodiment, the gas entering the membrane space 114 may be different at the initial stage to allow reaching an equilibrium faster. In yet another embodiment, the content of the gas may adjust over time by inserting a different mixture of its components. In such case, the Nitrogen Oxygen and carbon dioxide may be adjusted to closely mimic the concentration inside the body.

FIGS. 2A-2E show cross sectional views of the analyzing catheter at the indicated locations in FIG. 1. Shown here are catheter shaft 102, gas entry lumen 104, gas exit lumen 106, gas entry opening 108, gas exit opening 110, membrane 112 membrane space 114, and catheter inner lumen 202. The analyzing catheter may or may not have an inner lumen. The membrane space may be maintained by a positive pressure created by the gas pumped into the space and/or the space may be maintained by structures, such as rings, hoops, or scaffolding etc. In yet another embodiment, the pump is designed to create a negative pressure that will help extracting the gas from the catheter. In such case, the risk of introducing unwanted gas into the body via the catheter is eliminated. In such case, a one way valve 103 may be places at the tip of the catheter to eliminate any liquids entering the membrane cavity 105 due to the application of the negative pressure but still allowing liquids to flow into the body 107.

FIG. 1 shows gas entry lumen 104 and gas exit lumen 106 extending past the membrane. In some embodiments one or both lumens may terminate more proximally or more distally.

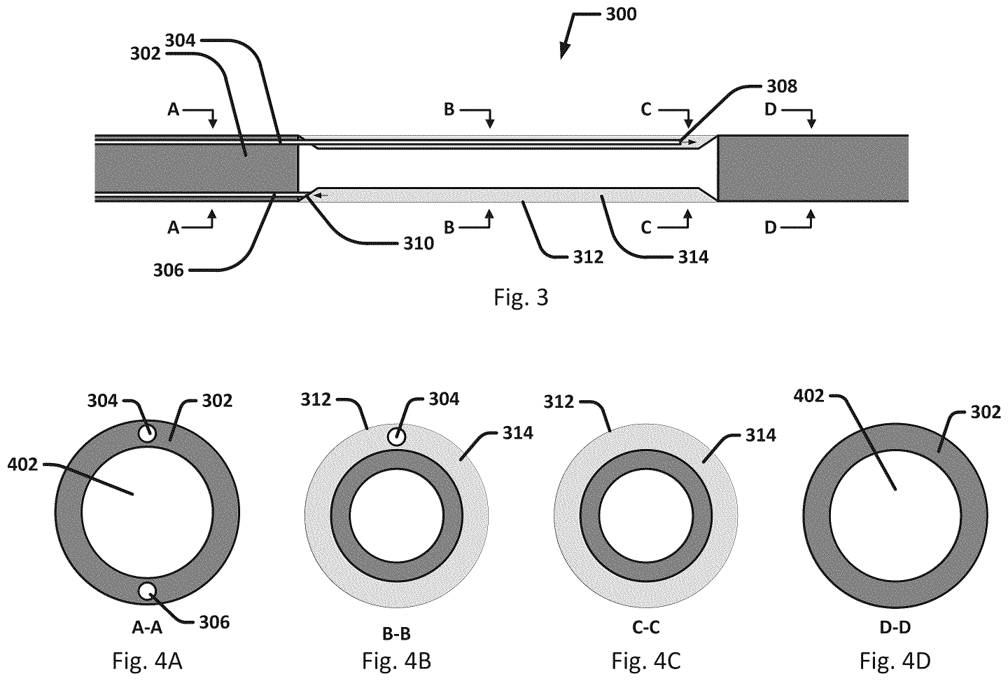

FIG. 3 shows another embodiment of the analyzing catheter. This embodiment includes a recessed section of the catheter shaft so that the membrane section diameter is flush or substantially flush with the rest of the catheter. Analyzing catheter 300 includes catheter shaft 302, gas entry lumen 304, gas exit lumen 306, gas entry opening 308, gas exit opening 310, membrane 312 and membrane space 314. Gas, such as a neutral gas, or air, is intruded into membrane space 314 via entry lumen 304 and entry opening 308. The gas within membrane space 314 is exposed to fluid or tissue in the area of the membrane (i.e. blood, in a blood vessel). Compounds which are small enough to cross the membrane cross from the blood or fluid or tissue into annular membrane space 314. The concentration of the compound will equilibrate across the membrane. The gas containing the compound is extracted via gas exit opening 310 and gas exit lumen 306 and analyzed to determine the concentration of the compound in the gas. From the information the controller may determine the concentration of the compound in the bodily fluid and/or tissue, such as the blood. The concentration of the compound may be the same in the extracted gas and the blood, or it may be different in a predictable way so that the controller can determine the concentration of the compound in the blood.

FIGS. 4A-4D show cross sectional views of the analyzing catheter at the indicated locations in FIG. 3. Shown here are catheter shaft 302, gas entry lumen 304, gas exit lumen 306, gas entry opening 308, gas exit opening 310, membrane 312 membrane space 314, and catheter inner lumen 402. The analyzing catheter may or may not have an inner lumen. The membrane space may be maintained by a positive pressure created by the gas pumped into the space and/or the space may be maintained by structures, such as rings, hoops, or scaffolding etc.

The gas membrane of the analyzing catheter may comprise part of all of the length of the catheter. The membrane section may be flush with, have a larger diameter than, have a smaller diameter than, the rest of the catheter shaft.

The gas entry opening and the gas exit opening may be far enough away from each other to allow equilibration of the gas within the membrane space with the fluid and/or tissue outside the membrane. For example, the distance between the two openings may be greater than 1 mm. Or, for example, the distance between the two openings may be greater than 2 mm. Or, for example, the distance between the two openings may be greater than 5 mm. Or, for example, the distance between the two openings may be greater than 10 mm. Or, for example, the distance between the two openings may be greater than 20 mm. Or, for example, the distance between the two openings may be greater than 30 mm. Or, for example, the distance between the two openings may be greater than 40 mm.

The gas entry opening and the gas exit opening may be 180 degrees from each other to maximize gas mixing. The membrane space may include baffles or guides to guide the gas between the entry opening and the exit opening to maximize gas mixing.

In yet another embodiment, the membrane can be semipermeable and acting as a microdialyzer. Made of material suOfine polyurethane and acting as an artificial “blood capillary”. The permeation can happen due to osmosis, electrical gradient or hydolic pressure. The pump can then perfuse the catheter with saline and or solution containing Na+, K+, Ca+, Mg2+, and Cl−. Once the fluid passes through the inner tube of the catheter and reaches the membrane area, the chemical substances at higher concentration in the extracellular fluid (ECF) passively diffuse into the perfusate driven by the concentration gradient, thus allowing sampling of amino acids and small proteins the extracted fluids can be analyzed by the analyzer a PCR reader, colorimeter and or spectrophotometer., or collected such as in a vial and analyzed on an external device. Such system can be used to detect virtually any substance that is small enough to cross the membrane can be measured with an appropriate analytic technique such as Animo acids, or measure common biochemical markers such as glucose, lactate, pyruvate, glutamate, glycerol, and urea. Alternatively, a retrodialysis can be implemented wherein chemical substances can also be added to the perfusion fluid and will diffuse across the membrane into the ECF. In such case, the system can be used also as a delivery mechanism of pharmacologic and experimental agents directly into tissue. As the concentration of gas such as carbon dioxide is a function of the temperature, the catheter may also include means of measuring core body temperature.

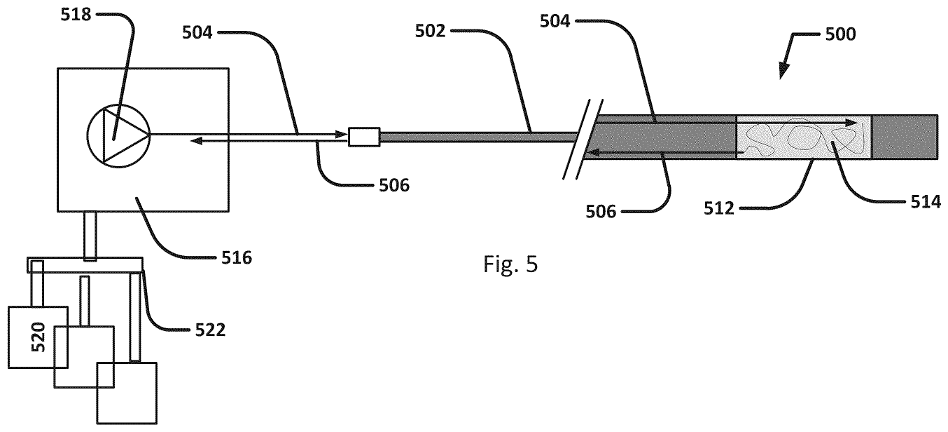

FIG. 5 shows the analyzing catheter system. Shown here is analyzing catheter 500, gas entry lumen 504, gas exit lumen 506, membrane 512, membrane space 514, controller 516 and pump 518. The controller controls the flow of gas into and/or out of the membrane space via pump 518. The system may also include a gas reservoir for the entry gas and/or a gas reservoir for the exit gas. Such reservoir may include a plurality of different gas containers, 520 such as air, nitrogen, oxygen, carbon dioxide, and a mixer 522 that can provide either inert gas such as nitrogen, or mixed air in different percentages to correspond to the gasses in the blood. The system may also include the ability to analyze the exit gas for concentration of the component or components. The system may also include the ability to flush the entry lumen, exit lumen, membrane and/or membrane space. The analyzer can be specific to certain gasses, using colorimetry, enzyme reaction, PCR or even a generic spectrophotometer to allow multi component analyzer. Alternatively, rather than an analyzer, the exit lumen may be connected to a sample collection, such as a vile, which will fill with the compound and be analyzed externally.

### Example of Data Processing System

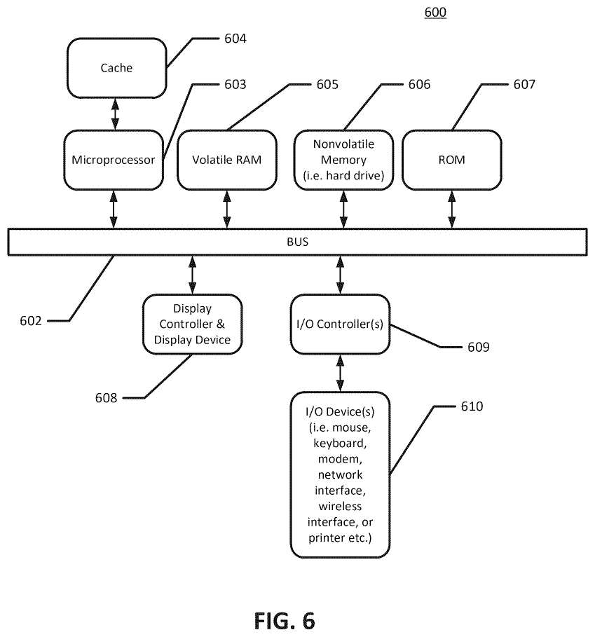

FIG. 6 is a block diagram of a data processing system, which may be used with any embodiment of the invention. For example, the system 600 may be used as part of a controller/monitor disclosed herein. Note that while FIG. 6 illustrates various components of a computer system, it is not intended to represent any particular architecture or manner of interconnecting the components; as such details are not germane to the present invention. It will also be appreciated that network computers, handheld computers, mobile devices, tablets, cell phones and other data processing systems which have fewer components or perhaps more components may also be used with the present invention.

As shown in FIG. 6, the computer system 600, which is a form of a data processing system, includes a bus or interconnect 602 which is coupled to one or more microprocessors 603 and a ROM 607, a volatile RAM 605, and a non-volatile memory 606. The microprocessor 603 is coupled to cache memory 604. The bus 602 interconnects these various components together and also interconnects these components 603, 607, 605, and 606 to a display controller and display device 608, as well as to input/output (I/O) devices 610, which may be mice, keyboards, modems, network interfaces, printers, and other devices which are well-known in the art.

Typically, the input/output devices 610 are coupled to the system through input/output controllers 609. The volatile RAM 605 is typically implemented as dynamic RAM (DRAM) which requires power continuously in order to refresh or maintain the data in the memory. The non-volatile memory 606 is typically a magnetic hard drive, a magnetic optical drive, an optical drive, or a DVD RAM or other type of memory system which maintains data even after power is removed from the system. Typically, the non-volatile memory will also be a random access memory, although this is not required.

While FIG. 6 shows that the non-volatile memory is a local device coupled directly to the rest of the components in the data processing system, the present invention may utilize a non-volatile memory which is remote from the system; such as, a network storage device which is coupled to the data processing system through a network interface such as a modem or Ethernet interface. The bus 602 may include one or more buses connected to each other through various bridges, controllers, and/or adapters, as is well-known in the art. In one embodiment, the I/O controller 609 includes a USB (Universal Serial Bus) adapter for controlling USB peripherals. Alternatively, I/O controller 609 may include an IEEE-1394 adapter, also known as FireWire adapter, for controlling Fire Wire devices.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques shown in the figures can be implemented using code and data stored and executed on one or more electronic devices. Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals-such as carrier waves, infrared signals, digital signals).

The processes or methods depicted in the preceding figures may be performed by processing logic that comprises hardware (e.g. circuitry, dedicated logic, etc.), firmware, software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

All embodiments disclosed herein may incorporate features from other embodiments disclosed herein.

The applications of the devices and methods discussed above are not limited to the fields described but may include any number of further applications in other fields. Modification of the above-described assemblies and methods for carrying out the invention, combinations between different variations as practicable, and variations of aspects of the invention that are obvious to those of skill in the art are intended to be within the scope of the claims.

1. A catheter based system for analyzing one or more components in a body, comprising:
a catheter having a length;
one or more membranes positioned along the length of the catheter, wherein the one or more membranes are configured to diffuse one or more compounds from a fluid, bodily gas, or tissue of interest through the one or more membranes and into a membrane space defined by the at least one membrane;
a gas introduction lumen in fluid communication with the membrane space for introducing a gas through the length of the catheter and into the membrane space;
a gas extraction lumen in fluid communication with the membrane space for extracting the gas and the one or more compounds dispersed within the gas from the membrane space; and
an analyzer in communication with the membrane space, wherein the analyzer is configured to determine a parameter of the one or more compounds.

2. The system of claim 1 further comprising a pump in communication with the gas introduction lumen.

3. The system of claim 1 wherein the one or more membranes are configured for contacting blood.

4. The system of claim 1 wherein the one or more membranes are configured to be selectively permeable to one or more types of compounds.

5. The system of claim 4 wherein the one or more types of compounds comprise oxygen, carbon dioxide, or nitrogen.

6. The system of claim 1 wherein the analyzer is configured to determine a concentration of the one or more compounds.

7. The system of claim 1 wherein the analyzer is configured to determine information relating to arterial blood gases.

8. The system of claim 1 further comprising at least one additional membrane which defines the membrane space.

9. The system of claim 1 wherein the membrane space defines an annular space within or around the catheter.

10. The system of claim 9 wherein the annular space is defined through the length of the catheter from the membrane space and to the analyzer.

11. The system of claim 2 wherein the pump is configured to form an adjustable negative pressure within the membrane space.

12. The system of claim 11 further comprising a one-way valve near or at a tip of the catheter, wherein the valve is configured to prevent gas from entering the body.

13. The system of claim 2 wherein the pump is configured to pump the gas through the length of the catheter and into the membrane space.

14. The system of claim 13 wherein the gas comprises an inert gas.

15. The system of claim 13 wherein the pump is configured to pump air with adjustable oxygen and nitrogen concentration

16. The system of claim 1 wherein the one or more membranes comprises a directional membrane configured to allow for unidirectional movement of the one or more compounds through the one or more membranes and into the membrane space.

17. The system of claim 16 wherein the directional membrane is configured to allow for bidirectional movement of the one or more compounds.

18. The system of claim 1 further comprising one or more sensors positioned along the catheter and configured to measure pH or core temperature.

19. A method of analyzing one or more components in a body, comprising:
positioning a catheter into contact with a fluid or tissue of interest within the body, wherein the catheter comprises one or more membranes which defines a membrane space within;
introducing a gas through a length of the catheter and into the membrane space such that one or more compounds which have diffused through the one or more membranes from the fluid or tissue of interest and into the membrane space are dispersed within the gas;
extracting the gas having the one or more compounds from the membrane space and into an analyzer in fluid communication with the membrane space; and
determining a parameter of the one or more compounds via the analyzer.

20. The method of claim 19 wherein positioning the catheter into contact with the fluid or tissue of interest comprises positioning the one or more membranes into contact with blood.

21. The method of claim 19 wherein the one or more membranes are configured to be selectively permeable to one or more types of compounds.

22. The method of claim 21 wherein the one or more types of compounds comprise oxygen, carbon dioxide, or nitrogen.

23. The method of claim 19 wherein determining the parameter comprises determining a concentration of the one or more compounds.

24. The method of claim 19 wherein determining the parameter comprises determining information relating to arterial blood gases.

25. The method of claim 19 wherein the catheter further comprises at least one additional membrane which defines the membrane space.

26. The method of claim 19 wherein the membrane space defines an annular space within or around the catheter.

27. The method of claim 26 wherein the annular space is defined through a length of the catheter from the membrane space and to the analyzer.

28. The method of claim 19 wherein introducing the gas further comprises adjusting the concentration of the gas until the one or more compounds are in equilibrium through the one or more membranes.

29. The method of claim 19 wherein extracting the gas having the one or more compounds comprises withdrawing the gas via an adjustable negative pressure formed within the membrane space to prevent gas entering the body through said catheter.

30. The method of claim 19 wherein introducing the gas through the length of the catheter comprises pumping the gas into the membrane space.

31. The method of claim 30 wherein pumping the gas into the membrane space comprises pumping air in adjustable concentration of oxygen, carbon dioxide, and nitrogen into the membrane space.

32. The method of claim 19 wherein the one or more membranes comprises a directional membrane configured to allow for unidirectional movement of the one or more compounds through the one or more membranes and into the membrane space.

33. The method of claim 32 wherein the directional membrane is configured to allow for bidirectional movement of the one or more compounds.

34. The method of claim 19 further comprising sensing core temperature via one or more sensors positioned along the catheter.

35. The method of claim 19 further comprising sensing a pH via one or more sensors positioned along the catheter.

36. The method of claim 19 further comprising sensing systolic and diastolic blood pressure via one or more sensors positioned along the catheter.

37. The method of claim 19 where a pump temporally alternates between introducing a gas and extracting a gas.

38. The method of claim 19 wherein gas contains one or more elements that interact with the measured component or other components.
